(19) United States
(12) Patent Application Publication
(10) Pub. No.: US 2013/0060160 A1
(43) Pub. Date: Mar. 7, 2013

(54) TISSUE AND FLUID SAMPLING DEVICE AND METHOD

(71) Applicant: Stephen Heier, Briar Cliff, NY (US)

(72) Inventor: Stephen Heier, Briar Cliff, NY (US)

(21) Appl. No.: 13/492,988

(22) Filed: Jun. 11, 2012

Related U.S. Application Data
(60) Provisional application No. 61/530,001, filed on Sep. 1, 2011.

(51) Int. Cl. A61B 5/15; A61B 5/153; A61B 10/02

(52) U.S. Cl. 600/566; 600/562; 600/573; 600/578; 600/575

(57) ABSTRACT

A device and method for acquiring tissue or fluid. The device includes an instrument for acquiring tissue or fluid, and a pump. The instrument has a proximal end and a distal end at which the tissue or fluid is first acquired. The pump is connected to the proximal end of the instrument for applying negative pressure for drawing in the tissue or fluid. The pump has a switch for adjusting the amount of pressure. The method includes inserting an instrument into the site of the tissue or fluid to be acquired and exerting a controlled amount of adjustable negative pressure through the instrument to draw back into or onto the instrument the tissue or fluid being acquired.

This application claims the benefit under 35 U.S.C. 119(e) of U.S. provisional Application No. 61530001, having a filing date of Sep. 01, 2011.

### FIELD OF INVENTION

The present invention relates to a device and method using adjustable negative pressure for tissue and fluid acquisition.

### BACKGROUND OF THE INVENTION

Fine needle aspiration, or FNA, is a commonly used technique to obtain tissue or fluid specimens from the human body, by inserting a fine small caliber needle into the targeted tissue, and then aspirating or sucking back through the needle to withdraw the desired specimen. Typically, the negative suction is either applied at will by the operator pulling back on a syringe connected to the hub of a needle, or a small syringe delivering a set negative pressure (on pull back with a set lock) is attached to the hub of the needle with its suction opened up to the needle at the appropriate time after the needle enters the targeted tissue.

This current methodology has a number of inherent drawbacks. On the one hand, when operator syringe control is used to maintain the amount of suction applied, the process is awkward at best while this same operator is thrusting the syringe and its attached needle in and out of the tissue targeted. Also, this technique imposes certain limitations on the amount and regulation of suction during FNA. On the other hand, a set negative pressure syringe limits the operator's ability to respond to the tissue characteristics during FNA. Furthermore, it is impossible to regulate the negative pressure, as any tissue or fluid withdrawal effectively lessens the pressure for additional tissue acquisition, and any loss of integrity in the system may release some or all of the negative pressure without awareness by the operator, resulting in continued risk during the procedure without the perceived benefit.

### SUMMARY OF THE INVENTION

The device and method of the invention overcomes all of these drawbacks as discussed above (in “Background of the Invention”, providing ready regulation of the negative pressure, as well as fully adjustable and variable negative pressure, and from a distance that does not interfere with the operator's performance of the FNA. Furthermore, in the case of fluid acquisition, it provides for direct aspiration into a receptacle that allows for ready transport and processing without specimen loss or contamination.

The device and method of the invention acquires tissue or fluid using an instrument for acquiring tissue or fluid. The instrument has a proximal end and a distal end at which the tissue or fluid is first acquired. A pump is connected to the proximal end of the instrument for applying negative pressure for drawing in the tissue or fluid.

The inventive method of acquiring tissue or fluid comprises: inserting an instrument into the site of the tissue or fluid to be acquired; and exerting a controlled amount of negative pressure through the instrument to draw back into or onto the instrument the tissue or fluid being acquired.

### BRIEF DESCRIPTION OF THE DRAWINGS

The present invention will be more readily understandable from a consideration of the accompanying drawings in which:

FIG. 1 depicts a hand held pump;

FIG. 2 depicts a needle;

FIG. 3 depicts the hand held pump connected to the needle; and

FIG. 4 depicts the device of FIG. 3 to which has been added a fluid collection chamber.

### DETAILED DESCRIPTION OF THE INVENTION

The inventive device is an applicable aid to the broad range of FNA and suction specimen retrieval procedures. For example, it is suitable for Endoscopic Ultrasonography (EUS) FNA, for Cystoscopy, Bronchoscopy and Laparoscopy guided tissue acquisition, for CT (computed axial tomography) scan and ultrasound guided FNA, for liver biopsies, for Paracentesis and Thoracentesis, and for any other similar tissue or fluid acquisition where suction or negative pressure is utilized and specimen capture is required.

It is beneficial for the suction to have a variable nature, as illustrated by contrasting two extremes in specimen acquisition. At one extreme, if a tissue is known to be extremely vascular by its Doppler or other characteristics, or is found to be extremely vascular by a bloody specimen return, the amount of suction can be easily dialed down to reduce tissue damage and loss of specimen integrity. At the other extreme, if a certain volume of non-bloody cyst fluid is to be sampled, the amount of suction and negative pressure can be easily pumped or dialed up, allowing for more rapid fluid withdrawal. This is especially useful when the first fluid being obtained reduces the effective negative pressure for continued success. In both of these extreme instances, the inventive device and method allows for a safer and interactive mechanism that is easy to control, with high quality tissue preservation for easier pathologic examination, and with shorter operator times and possibly fewer invasive sticks to the patient.

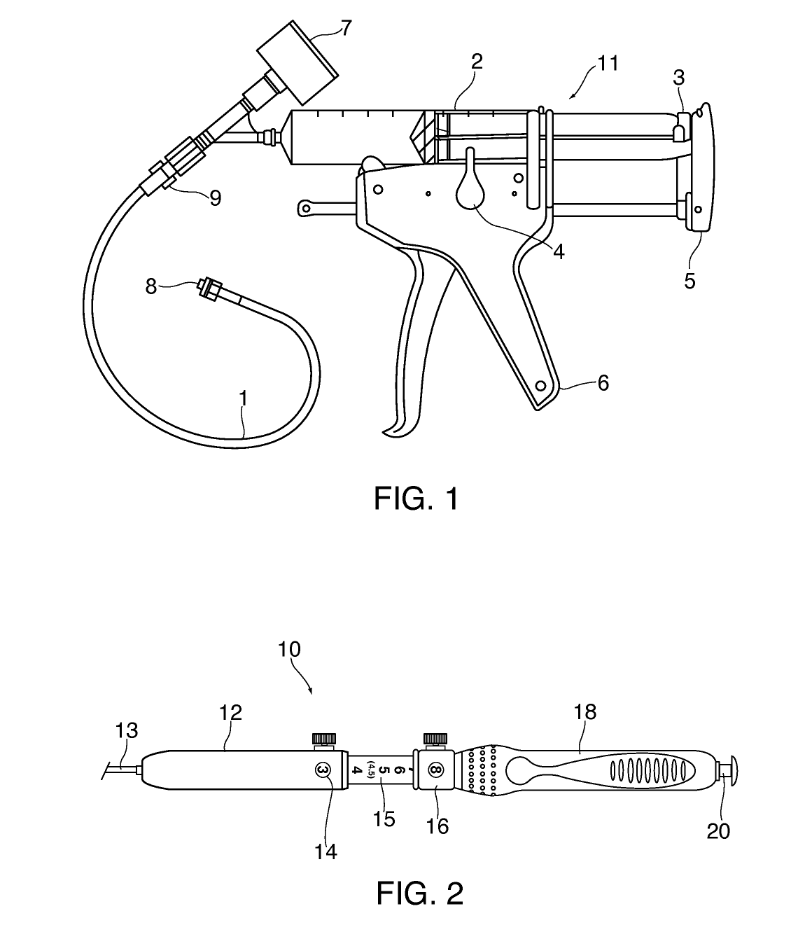

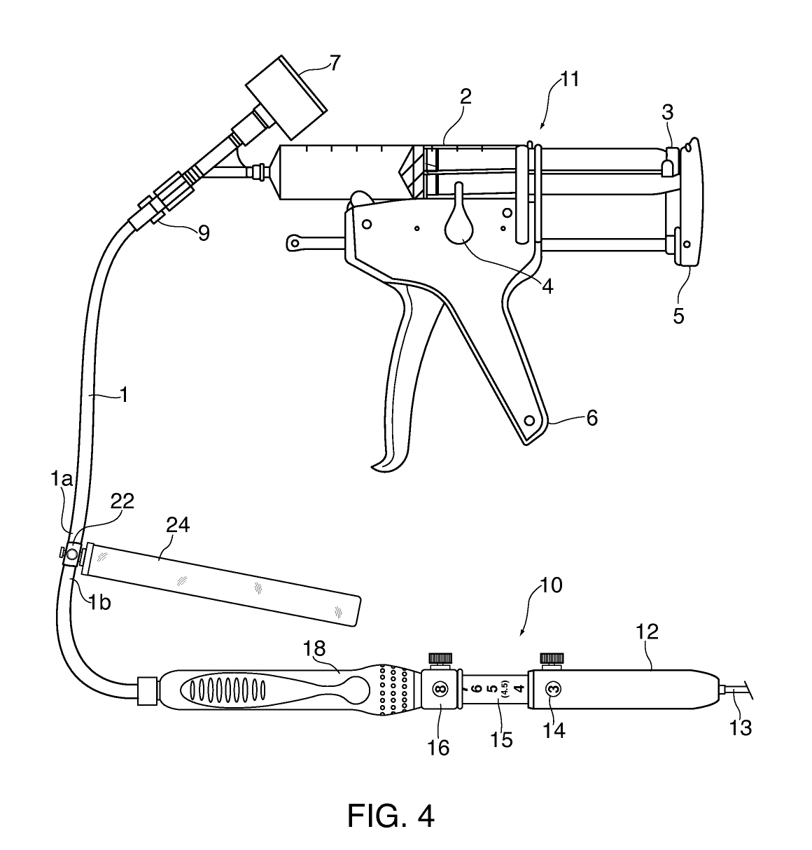

The inventive device can be divided into several components: a hand held vacuum gun 11, as shown in FIG. 1 (which can be used with or without a negative pressure gauge 7), connecting tubes 1 with conventional stopcocks and pinchers to allow distance so as not to interfere with the operator's performance of the fluid or tissue acquisition and to isolate the captured specimen, an instrument for acquiring the fluid or tissue, and an optional specimen receptacle 24, in FIG. 4, that interposes between connecting tubes 1a and 1b for fluid capture, transport and processing. The specimen receptacle is not needed for solid tissue acquisition, since a solid specimen is retained in the needle's luminal channel core, from where it can be forward expelled after needle retrieval from the instrument used to reach the tissue. This specimen is generally forced back out with a stylet 20, in FIG. 2, that passes through the hub of the needle device down to the tip of the needle, or by blowing the specimen out of the needle with a burst of air or fluid delivered through the hub of the needle device. The specimen receptacle is therefore an optional component, and may also not be needed for the acquisition of very small quantities of fluid that are unlikely to fill the length of the needle's luminal channel core.

The hand held vacuum (or inflation) gun can be the same or similar to the Alliance II Inflation System from Boston Scientific, which is depicted in FIG. 1. However, the gun is used functionally in the exact opposite manner that was intended. The developed use of this gun is to blow fluid or air into balloons that are connected by a catheter to the gun. The balloons are used to stretch narrowings in the gastrointestinal tract, so as to stretch scar tissue and strictures that block the intestinal tract. By stretching these blockages with a balloon, normal intestinal tract patency can be restored so as to allow, for example, passage of a food bolus.

As shown in FIG. 1, the vacuum gun has a connecting or extension tube 1, a syringe/gauge assembly 2, a heel clip 3, a directional switch 4, a heel plate 5, an inflation/deflation handle 6, and a pressure gauge 7.

In the device and method of the invention, this inflation gun is instead used as a vacuum gun with negative pressure. Rather than stretching apart a narrowing by pumping air or fluid under positive pressure into a balloon system, the vacuum gun is used to create a significant, measurable, and tunable negative pressure, by withdrawing air into the system from an otherwise neutral zone. The directional switch on the gun allows for pressure to be decreased further or increased at will, by either pulling more out of the targeted zone, or by releasing some of the negative pressure.

The amount of negative pressure will be dependent on the number of cc's backwards pressure exerted by the vacuum gun, the tissue or fluid characteristics, and other technical factors. In one example, the amount of negative pressure is configured in increments from 0 to 60 cc's. The amount of negative pressure used in real time is continuously measured and under the watch of the operator or an assistant, and therefore can always be known to and directed by the operator, rather than being a mystery of the system.

The vacuum gun can be used with a continuous range of negative pressures, correlating with 0 to 60 cc of backwards suction pressure on a syringe. Usually, 30 cc of pressure is used, but ranges of pressure are usable, such as, approximately, 0-10 cc, 10-20 cc, 20-30 cc, 30-40 cc, 40-50 cc, and 50-60 cc. The range or maintenance of pressure used depends on the sample to be obtained. For example, where the tissue sample has a high degree of vascularity, less pressure, e.g., less than 30 cc, is used so the sample is less bloody and the amount of pressure is maintained throughout the collecting of the sample. On the other hand, when the sample is a large volume of fluid, a higher pressure may be used, e.g., more than 30 cc.

The Connecting Tubing 1 is depicted in the figures. As shown in FIG. 1, stop cock 9 (end of connecting tube 1) connects to the vacuum gun, and the other end is a connector 8 which connects to the proximal end of an instrument for acquiring fluids or tissue. This connecting tubing can have a stopcock at either or both ends based on convenience, so that the negative pressure can be easily “turned on” or “turned off” totally, as opposed to scaled up and down by the vacuum gun. Also, the tube comes with pinchers, so that captured fluid can be more readily trapped in a certain length of tubing without dripping when disconnected and without fluid loss by being exposed to and coating unnecessary parts of the connecting tubing. The pincher is attached along the length of the connecting tubing. It functions basically like a bobby pin that pinches off the tubing when you slide it fully across the tubing. But is slightly different from a bobby pin in that it is closed at both ends so it won't fall off the tubing and get lost. It also looks less like a bobby pin & more like a flat piece of rectangular plastic with an old style-like key hole cut out of the center. The circle of the key hole is where the tube would normally traverse without being pinched or impeded, and the tapering line below the circle where you would push the tube into to occlude that section. Since the pincher can slide along the tube, it can be used to pinch off the tube any place along the tube where it may be most advantageous.

The instrument for acquiring fluids or tissue can be a needle, such as that used in fine needle aspiration. In one example, the instrument 10 has an outside handle 18 and the connecting tubes 1 can be connected to the hub of that handle, as shown in FIG. 2. The handle 18, in turn, leads to a needle (not shown) that is protected by a catheter 13 that can pass through an endoscope or other device to reach the targeted tissue prior to needle exposure and tissue insertion. In the needle device 10, a lock keeps the needle from coming out until it is needed by pulling out the stylet 20 as in any fine needle aspiration procedure.

Normally, a needle would be put directly on top on the specimen to suck back on it or set into suck back mode using the bulb. The syringe would be attached at the top to a tube which is attached to the bulb to suck back a certain amount of the specimen. An alternative is to use a bulb having 10 cc pressure, but it is inefficient to use to obtain a tissue or fluid specimen culture through the scope. It would, instead, be necessary to use another device to obtain a tissue or fluid specimen culture, or to repeat the process, or just not be able to get a sufficient amount of a specimen.

The needle used to obtain the tissue or fluid specimen can be any of a variety of endoscopic or percutaneous tissue or fluid acquisition needles, that can be hooked up to the hand held pump through universal connectors. The specimen itself is collected within the lumen of the needle or its attached hollow cable, or within the tubing of the device, its tissue or fluid collecting chamber, or the syringe, depending on the volume of the specimen obtained. The needle extends from a catheter and the needle on the sheath as well as the catheter are hollow to contain the needle and aspirate through the center of the catheter. A collection chamber is not needed for solids. The solid tissue specimen remains in the needle after collection. Then, the specimen is removed by pushing the stylet 20 back into the needle device 10 or blowing out the needle with an air-filled syringe.

As shown in FIG. 2, is needle device 10 which is used for fine needle aspiration procedures. The needle device shown in FIG. 2 is a Cook Medical Echotip Ultra Celiac Plexus Neurolysis Needle. The needle device 10 in FIG. 2 has a sliding sheath adjuster 12, a sheath reference mark 14, a center section 15 with alternative reference marks, a safety ring 16, a needle handle 18 and a stylet hub 20. At the opposite end from the stylet hub 20, the needle device 10 includes a fine needle shown in its own covering sheath 13.

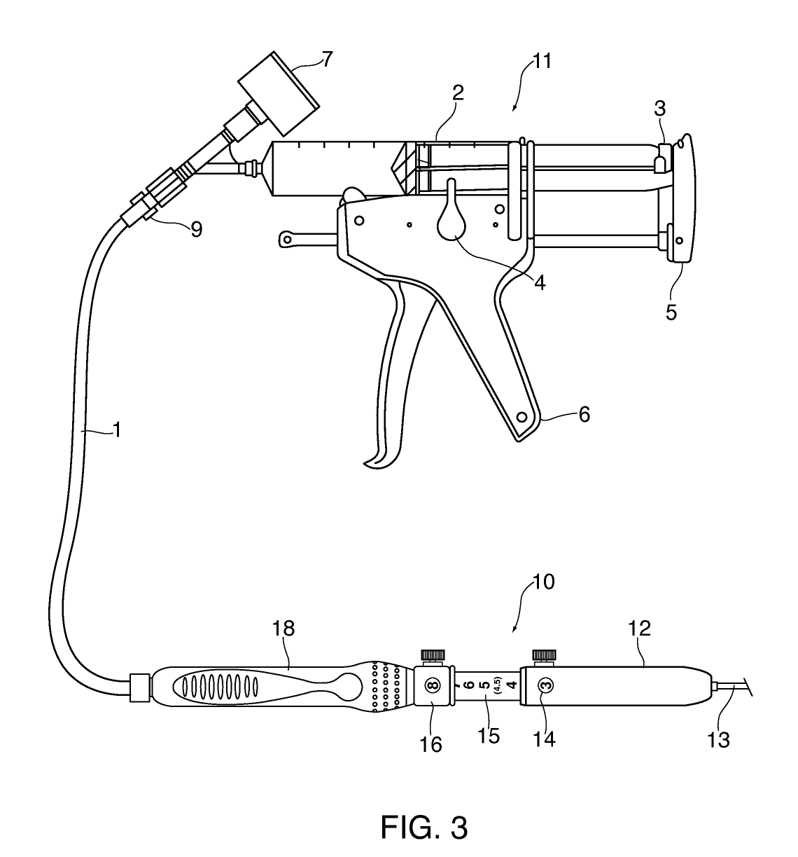

FIG. 3 shows the inventive device, which is used in the method of the invention, including the hand held pump 11 and the needle device 10, where the stylet hub 20 has been removed from the needle device 10 and the extension tubing 1 of the hand held pump 11 is connected to the needle device 10 at the site where the stylet hub 20 has been removed.

The pump can be fitted with a pressure gauge 7 to set the amount of suction and keep it constant (maintain it) and monitor it throughout the procedure irrespective of the amount of fluid or tissue being pulled back. In the invention it is also possible to use a variable amount of suction during the procedure. Using a suction gauge 7 is beneficial, since sometimes there is a loss of pressure when performing a fine needle aspiration and the operator does not even know the pressure has changed. This suction gauge 7 helps to both set the degree of suction and maintain that degree of suction.

Finally, the inventive device can have, as shown in FIG. 4, an optional specimen receptacle 24 that interposes a stopcock connecting device 22 between two connecting tubes 1a and 1b for fluid capture, collection, transport and processing. The specimen receptacle 24 allows for transmission of the negative pressure, but with the fluid drawn into the receptacle being captured. This specimen receptacle or collection chamber 24 can be in the form of a test tube with two proximal connecting spouts on opposing sides, one for fluid entry and the other for suction transmission, with a test tube stopper on top to allow easy access for subsequent lab testing. The size of the test tube or collection chambers can be varied depending on the size of the fluid collection to be drained and the amount of anticipated fluid acquisition. Two plugs (not shown) are provided to close off the side spouts during transport, with a circumferential elastic or rubber band around the two plugged spouts to prevent the plugs from being dislodged. The use of a test tube as a fluid collection chamber, for transport and subsequent manipulation, avoids additional transfer which otherwise could lead to losing some of the sample.

What is claimed is:

1. A device for acquiring tissue or fluid comprising:
an instrument for acquiring tissue or fluid, the instrument having a proximal end and a distal end at which the tissue or fluid is first acquired;
a pump being connected to the proximal end of the instrument for applying negative pressure for drawing in the tissue or fluid; and
the pump having a switch for adjusting the amount of pressure.

2. The device of claim 1, wherein the instrument is a needle.

3. The device of claim 1, wherein the pump further comprises a handle for generating negative or positive pressure by hand.

4. The device of claim 1, further comprising a tube to connect the instrument to the pump.

5. The device of claim 4, further comprising another tube and a specimen receptacle, wherein the specimen is interposed between the two tubes.

6. The device of claim 1, further comprising a pressure gauge.

7. A method for acquiring tissue or fluid comprising:
inserting an instrument into the site of the tissue or fluid to be acquired;
and exerting a controlled amount of adjustable negative pressure through the instrument to draw back into or onto the instrument the tissue or fluid being acquired.

8. The method of claim 7, wherein the pressure is exerted by a pump and the pump has a handle, and further comprising adjusting the pressure by squeezing the handle.

9. The method of claim 7, further comprising a step of regulating the pressure while acquiring the tissue or fluid.

10. The method of claim 7, further comprising a step of monitoring the pressure using a pressure gauge.

11. The method of claim 7, wherein the method is used together with endoscopic ultrasonography FNA, Cystoscopy, bronchoscopy, laparoscopy guided tissue acquisition, computed axial tomography scan, ultrasound guided FNA, liver biopsies, paracentesis or thoracentesis.

12. The method of claim 7, wherein the negative pressure being exerted is a range approximately between 0-10 cc, 10-20 cc, 20-30 cc, 30-40 cc, 40-50 cc, or 50-60 cc.

13. The method of claim 7, further comprising a final step of exerting positive pressure to release the fluid or tissue which has been acquired.
